(19) United States
(12) Patent Application Publication
(10) Pub. No.: US 2012/0330386 A1
(43) Pub. Date: Dec. 27, 2012

(54) SUBSTRATE PATTERNING

(75) Inventor: Andrew McNeill, Cellardyke (GB)

(73) Assignee: Ambicare Health Limited, St. Andrews, Fife (GB)

(21) Appl. No.: 13/512,897

(22) PCT Filed: Dec. 2, 2010
(86) PCT No.: PCT/GB10/52014

§ 371 Date: Sep. 11, 2012

(30) Foreign Application Priority Data
Dec. 1, 2009 (GB) 0920986.7

(51) Int. Cl. A61N 5/06

(52) U.S. Cl. 607/90

(57) ABSTRACT

There is herein described light emitting apparatus. More particularly, there is herein described light emitting apparatus capable of adapting and/or conforming to a non-planar surface on a patient's body.

### FIELD OF THE INVENTION

The present invention relates to light emitting apparatus. More particularly, the present invention relates to light emitting apparatus capable of adapting and/or conforming to a non-planar surface on a patient's body.

### BACKGROUND OF THE INVENTION

Traditional light sources are typically rigid structures and are not flexible. Moreover, these types of traditional light sources contain electronics which further adds to their rigidity. Traditional light sources that do flex have been found to be prone to breaking (or disconnecting from electronics) when folded past a certain angle. Therefore, in applications where a flat light source is required to conform to a neighbouring surface, this lack of flex limits the degree of conformability.

Moreover, even though some prior art light sources exist these types of light sources only allow a certain amount of flexibility in one direction, but not in two. This is sufficient for illumination of small surfaces but not for larger surfaces that may have contours in many directions at different points. A light emitting surface that is highly flexible and can bend in different directions at different points on this surface is therefore required.

Light can be used to treat a wide variety of diseases. When light alone is used to treat a disease, the treatment is referred to as phototherapy. Light may be used in conjunction with a pharmaceutical in which case the treatment is called photodynamic therapy.

Phototherapy and photodynamic therapy can be used to treat a variety of skin and internal diseases. In photodynamic therapy, a light-sensitive therapeutic agent known as a photopharmaceutical is supplied externally or internally to an area of the body which is to be treated. That area is then exposed to light of a suitable frequency and intensity to activate the photopharmaceutical. A variety of photopharmaceutical agents are currently available. For example there are topical agents such as 5-aminolevulinic acid hydrochloride (Crawford Pharmaceuticals) and methylaminolevulinic acid (Metfix (Trade Mark), Photocure). Often, the drug is applied in a non-active form that is metabolised to a light-sensitive photopharmaceutical.

In photodynamic therapy, the primary technique for supplying light to the photopharmaceutical is to project light of a suitable wavelength from standalone light sources such as lasers or filtered arc lamps. These sources are cumbersome and expensive, and are therefore only suitable for use in hospitals. This leads to inconvenience for the patient, and high cost for the treatment. High light irradiances are needed in order to treat an acceptable number of patients per day (for the treatment to be cost effective) and to avoid unduly inconveniencing the patient.

In order to achieve an effective photodynamic or phototherapy treatment, it is important that the area to be treated must be illuminated evenly. This ensures a consistent dose of light is administered across the treatments area. Other prior art light sources that are flexible consist of individual illuminators that are point sources. These point sources could be light emitting diodes embedded in a substrate that exhibits some degree of flexibility. The point like illumination nature of these light sources makes them intrinsically unsuited for homogenous illumination of treatment surfaces. The embedded nature of these light sources also means that as these light apparatus are folded or flexed, the illumination homogeneity is further reduced due to redirection and realignment of the individual point sources.

It is an object of at least one aspect of the present invention to obviate or mitigate at least one or more of the aforementioned problems.

It is a yet further object of at least one aspect of the present invention to provide light emitting apparatus comprising a light source wherein said medical apparatus is capable of adapting and/or conforming in a number of different directions.

### SUMMARY OF THE INVENTION

According to a first aspect of the present invention there is provided a light emitting apparatus for use in therapeutic and/or cosmetic treatment, the apparatus comprising:

at least one substrate;

at least one light source disposed on the substrate;

at least a first and second weakened area on the at least one substrate; and

wherein the first and second weakened area have substantially different directions and the light emitting apparatus is capable of adapting and/or conforming to a non-planar surface. The apparatus may therefore be a medical light emitting apparatus which may be used for a variety of therapeutic and/or cosmetic treatments.

In particular, the light emitting apparatus may therefore be adaptable and/or conformable to and/or around a non-planar surface. The light apparatus may bend, flex and/or conform around a non-planar, substantially non-planar and/or curved surface. There may be two or more or a plurality of weakened areas.

Typically, the first and second weakened areas may diverge with respect to each other and may, for example, have an angle between them of about 10-170°, about 20-160°, about 30-150°, about 40-140°, about 50-130°, about 60-120°, about 70-110°, about 80-100° or about 90°. In preferred embodiments the first and second weakened areas may have an angle of about 90° between them and may therefore be substantially orthogonal to one another.

In particular embodiments, there may be a series of weakened areas in a single substrate. The series of weakened areas may comprise a series of substantially vertical and substantially horizontal weakened areas (i.e. the substantially vertical weakened areas may go substantially up and down the substrate and the substantially horizontal weakened areas may go side-to-side across the substrate). The substantially vertical weakened areas may be a series of continuous weakened areas spaced apart on an x-axis and the substantially horizontal weakened areas may be a series of discontinuous weakened areas spaced apart on a y-axis.

Typically, there may be a series or plurality of substantially vertical weakened areas which may extend substantially the whole length of the substrate but may have a remaining part that is not weakened. The substantially vertical weakened areas may be continuous. By leaving a small part of the length of the substrate non-weakened provides the substrate with enough structural strength to stay in one-piece i.e. a single piece. There may also be a series or plurality of discontinuous substantially horizontal weakened areas which may, for example, intersect and extend though the substantially vertical weakened areas. The substantially horizontal weakened areas may be shorter than the substantially vertical weakened areas.

There may be a series or plurality of substantially horizontal weakened areas intersecting and extending though the substantially vertical weakened areas along at least part or substantially the whole length of the substantially vertical weakened areas.

Typically, there may be a plurality of substantially vertical weakened areas extending from one side of the substrate to an opposite side. The plurality of substantially vertical weakened areas may be substantially parallel with one another. Therefore the multiple substrates do not need to be connected.

Typically, there may be a plurality of substantially horizontal weakened areas extending from one side of the substrate to an opposite side. There may be at least one or more or a plurality of inter-dispersed substantially horizontal weakened areas in a single line. The plurality of substantially horizontal weakened areas in a single line may therefore form a discontinuous ‘dash’-like pattern with the plurality of substantially horizontal weakened areas intersecting and extending though the substantially vertical weakened areas. There may be a series of rows of substantially horizontal weakened areas each in a form of a ‘dash’-like pattern. The rows of substantially horizontal weakened areas may alternate with adjacent rows of the substantially horizontal weakened areas; meaning that where there is a weakened area in a row then in the adjacent row this is a non-weakened area.

The weakened areas may be thinner areas on the substrate, perforated areas, partially folded areas or cut areas. The cuts may extend from one side of the substrate to the other and may therefore form a plurality of slits in the substrate.

The weakened areas may be arranged in such a way to promote and allow flexing.

The material forming the substrate may be pliable and may bend, flex and/or conform in itself. For example, a flexible material (e.g. a flexible polymeric material) such as a thermoplastic elastomer polymer may be used. The substrate can therefore fold in multiple directions at the same time.

The thickness of the substrate may be sufficient to provide support to the at least one light source and may also be thick enough to incorporate a diffusing member which may be separate and parallel. Typically, the substrate may have a thickness of about 0.01-15 mm, about 0.01-5 mm, about 0.01-2 mm or about 0.1-1 mm.

The substrate may comprise one, at least one or more or a plurality of light sources located within the areas defined by the weakened areas.

The areas defined by the weakened areas may be substantially rectangular or square shaped. The areas defined by the weakened areas may have an area of about 0.1 to 4 cm² or about 0.5 to 1 cm².

The substrate may also comprise a diffusing member which may mirror the shape of the original substrate including the shape of the weakened areas.

Light from the light source(s) may be easily distributed across the substrate in a substantially homogenous manner; this may be achieved via a diffusing structure.

The light sources may be of any suitable shape and may, for example, be substantially circular, triangular, square, rectangular, pentagonal, hexagonal or any combination thereof.

Preferably, the light sources and diffusers may be located on the substrate so that they do not touch one another as the flexible apparatus bends, flexes and/or conforms away from a planar position. Therefore, at least some or preferably all of the sides of the light sources do not touch when the flexible apparatus is substantially planar (i.e. flat) or bent, flexed and/or conformed.

The light emitting apparatus of the present invention may therefore provide light emitting apparatus which is capable of adapting and/or conforming to a non-planar, substantially non-planar or curved surface on, for example, any part of a human or mammal body such as on a foot, leg, torso, shoulder, arm, hand, finger, head or facial area of a patient. As the light emitting apparatus may be flexible in multiple directions this allows relatively large areas of a patient to be covered and treated as although the substrate material may be at least semi-rigid the light emitting apparatus as a whole may be flexible.

By allowing the light emitting apparatus to adapt and/or conform to a non-planar or curved surface allows the light emitting apparatus to be capable of providing even or substantially even illumination of a patient's skin, which is important for efficacious therapy. The present invention may therefore allow at least one, two or more, or a plurality of rigid or semi-rigid light sources located on the flexible substrate to adapt and/or conform to non-planar and/or curved surfaces on a patient.

The flexible apparatus may comprise a surface intended to make contact with a patient (i.e. a contact surface). This contact surface may be capable of bending, flexing and/or conforming around a non-planar, substantially non-planar or curved surface of, for example, a patient. Preferably, all or substantially all of the flexible apparatus is capable of making contact with a patient during use.

The flexible apparatus with the substrate comprising a light source, diffuser and light emitting surface may be capable of bending, flexing and/or conforming away from a planar position in any direction by less than about 5 degrees, less than about 10 degrees, less than about 15 degrees, less than about 20 degrees, less than about 25 degrees, less than about 30 degrees, less than about 35 degrees, less than about 40 degrees, less than about 45 degrees, less than about 50 degrees, less than about 55 degrees, less than about 60 degrees, less than about 65 degrees, less than about 70 degrees, less than about 75 degrees, less than about 85 degrees or less than about 90 degrees. Alternatively, the substrate with the light source(s) may be capable of bending, flexing and/or conforming away from a planar position in any direction by about 0-5 degrees, about 0-10 degrees, about 0-15 degrees, about 0-20 degrees, about 0-25 degrees, about 0-30 degrees, about 0-35 degrees, about 0-40 degrees, about 0-45 degrees, about 0-50 degrees, about 0-55 degrees, about 0-65 degrees, about 0-70 degrees, about 0-75 degrees, about 0-80 degrees, about 0-85 degrees or about 0-90 degrees. The bending, flexing and/or conforming may occur at multiple points on the substrate and in different directions.

The light emitting apparatus may comprise electronics for the operation of the apparatus and the at least one light source. The electronics may also be flexible and be capable of bending, flexing and/or conforming away from a planar position. The substrate may be a flexible PCB.

The substrate may also be designed to allow light from the at least one light source to exit from the light emitting apparatus and be used in medical applications such as therapeutic and/or cosmetic treatment. The light emitting apparatus may therefore be capable of distributing light over an area of a patient to be treated and preferably substantially evenly over an area of a patient to be treated.

The light source(s) may be located on the substrate and light may be distributed over the surface of the rigid substrate via a diffusing member.

The light source(s) may be intrinsic area emitters such as an organic light emitting diode. In this case each rigid substrate will be an individual organic light emitting diode connected via a flexible hinge. Alternatively, the entire device may be a single substrate with weakened features.

The flexible light emitting apparatus intended to make contact with a patient may have a surface area of about 0.1-10000 cm², about 0.1-250 cm², about 0.1-100 cm², about 1-100 cm² or about 5-250 cm². Alternatively, the flexible light emitting apparatus intended to make contact with a patient may have a surface area of greater than about 0.01 cm², greater than about 0.1 cm², greater than about 1 cm², greater than about 5 cm², greater than about 10 cm², greater than about 50 cm² or greater than about 1000 cm².

The apparatus may also comprise an adhesive sheet around at least part or the entire periphery of the flexible substrate. The adhesive sheet may provide an adhesive surface for attaching the light emitting apparatus to a patient.

The flexible apparatus may also comprise a transparent adhesive sheet that sits between the flexible apparatus and the skin. The adhesive sheet may provide an adhesive surface for attaching the light emitting apparatus to a patient.

The apparatus may also comprise a drug and/or chemical source capable of delivering drugs and/or chemicals to the area of the patient to be treated. The apparatus of the present invention may therefore be capable of performing a therapeutic and/or cosmetic treatment.

The present invention may therefore relate to light emitting apparatus capable of performing a therapeutic and/or cosmetic treatment on a human or animal patient using photodynamic therapy or phototherapy. In some situations the treatment may be said to be of therapeutic nature (e.g. skin cancer, serious acne) whereas in other situations the treatment may be said to be of cosmetic nature (e.g. minor amounts of acne, anti-aging treatments such as treatment of wrinkling).

The light source may be any suitable light source that may emit light over a desired wavelength. The light source may be provided as a light emitting layer. Typically, the light source may operate within a range of about 300-3000 nm, about 300-1500 nm, about 300-800 nm or. In particular embodiments the light source may operate in the visible region of the electromagnetic spectrum. Alternatively, the light source may operate in the ultraviolet or infrared wavelength regions of the electromagnetic spectrum.

The light sources may emit light substantially continuously over a pre-set period of time or may emit light discontinuously such as in a pulsed manner. In particular embodiments, the light from the light source may be pulsed with a period of at least about 10 ms, at least about 100 ms, at least about 1 s, at least about 10 s, at least about 10 ms, at least about 100 s, at least about 1,000 s or at least about 10,000 s.

In particular embodiments, the light source may, for example, be any suitable form of diode such as organic light-emitting diode or an inorganic light-emitting diode incorporated into, for example, a diffuser. Alternatively, the light source may be a fluorescent light source such as a fluorescent lamp. The fluorescent light source may be compact in shape. For example, this includes technology developed from back-lit display technology, LEDs and waveguides and diffusers.

The light emitting apparatus as a whole may have an optical power density of about 0.1-500 mW/cm², about 1-200 mW/cm² or about 5-50 mW/cm². The at least one light source during operation may operate at a substantially constant power or alternatively may be varied over a range of powers.

The apparatus may comprise a photochemical and/or a photopharmaceutical preparation in a drug and/or chemical source in the form of, for example, a layer.

The photochemical and/or photopharmaceutical preparation may be delivered to the area of the patient to be treated at appropriate pre-set times and/or may be controlled by a control unit. The photochemical and/or a photopharmaceutical preparation may be present in the form of, for example, a gel, ointment, cream or gauze soaked in a photodynamic therapy solution. Alternatively, or in addition the light emitting apparatus may be provided with a thin film impregnated with a photochemical and/or photopharmaceutical preparation. The photochemical and/or a photopharmaceutical preparation may comprise a drug and/or antiseptic capable of treating a patient which may be applied to a person in need thereof. Typically, the photochemical and/or photopharmaceutical may be transparent or substantially transparent or may become transparent or substantially transparent during use and emission of light.

In embodiments where the photochemical and/or photopharmaceutical may be transparent or translucent to the wavelength of the irradiating light, the resulting device may be readily applied without a separate step of applying a photochemical and/or a photopharmaceutical to a patient. In particular embodiments, the photochemical and/or photopharmaceutical may be covered with a peelable release medium. The photochemical and/or photopharmaceutical preparation may comprise an inactive compound which may be metabolised in vivo to an active compound. During use a suitable amount of the photochemical and/or photopharmaceutical preparation may be applied to an area of a patient to be treated.

Typical drugs and/or chemicals used in the present invention include but are not limited to precursor drugs such as ALA or Metfix (Trade Mark) or other photodynamic therapy agents.

In use, the drug and/or chemical layer may be placed over the top of the area of the patient to be treated. To facilitate treatment an electrolyte solution such as sodium chloride solution may be placed on the skin to improve the electrical contact.

A power source may be supplied to power the light source and/or the electronics on the flexible substrate. The power source may be small and compact and may be integrated into the whole apparatus therefore making the apparatus suitable for ambulatory treatment. The apparatus of the present invention may therefore be wearable and may be attached to a foot, leg, torso, shoulder, arm, hand, head or facial area of a patient.

The light emitting apparatus may comprise attachment means for attaching the device to a body part(s) of a human or animal. For example, the apparatus may comprise mechanical and/or adhesive means for attaching the device to a body part of a human or animal. In particular embodiments, the light emitting apparatus may therefore comprise a strap arrangement which may optionally comprise a fastening means and/or Velcro (Trade Mark) and/or an adhesive surface for attaching the apparatus to the patient.

The light emitting apparatus may be lightweight and portable. In particular embodiments the apparatus may be a totally self-contained portable unit and may comprise a self-contained power supply. The power supply may operate electronics in the device and the light source. The light emitting apparatus may be sufficiently portable to enable ambulatory treatment therefore allowing treatment during which a patient may move around. Treatment may therefore occur at home or at work and may be removed by a patient when necessary. This provides lower treatment costs as this avoids out-patient or in-patient stays in hospital. This provides the significant advantage that lower light levels may be used since exposure can occur for a longer period of time. This overcomes the problem of pain induced in some patients by high irradiances from conventional sources used in hospitals. Moreover, lower irradiance over a longer period of time may be more effective in photoinduced therapy as it gives more time for oxygen to diffuse to the region to be treated, and reduces photobleaching of the photopharmaceutical.

The at least one light source may also comprise a substrate layer such as a transparent or at least a substantially transparent substrate layer. Alternatively, the at least light source may comprise a translucent or at least a substantially translucent substrate layer. The substrate layer may function as a support layer for the light source and may allow the light to penetrate there through. The substrate layer may also function as a barrier layer too and may be selected to prevent oxygen and/or moisture from penetrating the light source. The substrate layer may be made from or comprise any one of or combination of suitable materials such as a silicone, plastics or polymer. Additional layers may also be present, including diffuser layers

The apparatus according to the present invention may be used in a range of phototherapies and photodynamic therapies. For example, the apparatus according to the present invention may be used in the treatment of cancer (e.g. skin cancer), acne, wrinkles, wound-healing, anti-aging and post-skin laser treatments such as found in cosmetic applications. By wound is meant any form of open or closed wound. Open wounds include but are not limited to: incisions or incised wounds; lacerations; abrasions; puncture wounds; penetration wounds; gunshot wounds; and ulcers (including diabetic derived ulcers). Closed wounds include but are not limited to: contusions; hemaomas; and crushing injuries, fungal infections, treatments of bones and tendons.

According to a second aspect of the present invention there is provided a light emitting apparatus for use in therapeutic and/or cosmetic treatment, the apparatus comprising:

at least one substrate wherein the at least one substrate is a transparent electrode which is capable of functioning as a light source;

at least a first and second weakened area on the at least one substrate; and

wherein the first and second weakened area have substantially different directions and the light emitting apparatus is capable of adapting and/or conforming to a non-planar surface.

The second aspect is similar to the first aspect and all relevant parts are incorporated from the first aspect. The weakening and use may therefore be as defined in the first aspect.

The difference is that in this embodiment, the substrate is itself a transparent (or at least substantially transparent) electrode and the weakened area may be formed in the substrate and optionally also an anode. The anode and the substrate may therefore be patterned in a similar manner to the patterning described in the first aspect.

The substrate and the anode may be in planar form and may be sandwiched together.

In particular embodiments, the weakened area may form at least one or more or a plurality of cut-away members which may be flexible. On the cut-away members there may be a conductive trace or a plurality of conductive traces which may be in the form of a network or web which extends across the whole or substantially the whole surface area of the cut-away member. The conductive trace may be located towards and/or adjacent the sides of the cut-away members which means that there is more light emitted from the sides than the middle of the cut-away members. The conductivity traces may be thin with a thickness of about 0.1 μm to 5 mm.

The conductive trace distributes current across the surface of an anode of the light emitting apparatus (e.g. OLED) thereby overcoming its intrinsically high sheet resistance and inability to distribute current across its surface. This results in more homogenous light output from a polymer in the light emitting apparatus. The present inventors have found that in a cut/patterned substrate the geometry of the electrode traces are preferably configured to fit the shape of the cut-away members e.g. the ‘legs’.

In particular embodiments, the conductive traces may extend along the length or substantially a majority of the length of the cut-away members and along the middle or substantially the middle section of the cut-away members. The conductive trace may extend in a straight line or other patterns (e.g. curved or webbed) are possible. The conductive trace pattern may alternatively be in the form of a honeycomb pattern. However, other conductive trace patterns that form a network or web extending over the cut-away member may be used. It is preferred that the conductive trace pattern in the form of a network or web extends across the whole surface area of the cut-away member.

In alternative embodiments, the conductive trace extends down adjacent to both sides of the cut-away member and may then extend adjacent to the end of the cut-away member. The conductive trace pattern may therefore be seen as forming a substantially ‘U’-shaped design. The conductive trace pattern may be about 0.5-2 mm away from the edge of the cut-away member. By positioning the conductive traces towards and/or adjacent the sides of the cut-away members means that there is more light emitted from the sides than the middle of the cut-away members. This means that when two cut-away members are bent apart from each other, the adjacent surface is still illuminated evenly.

According to a third aspect of the present invention there is provided a method of performing a therapeutic and/or cosmetic treatment, the method comprising:

providing at least one substrate;

providing at least one light source disposed on the substrate;

providing at least a first and second weakened area on the at least one substrate; and

wherein the first and second weakened area have substantially different directions and the light emitting apparatus is capable of adapting and/or conforming to a non-planar surface.

According to a fourth aspect of the present invention there is provided a light emitting apparatus for use in therapeutic and/or cosmetic treatment, the apparatus comprising:

providing at least one substrate wherein the at least one substrate is a transparent electrode which is capable of functioning as a light source;

providing at least a first and second weakened area on the at least one substrate; and

wherein the first and second weakened area have substantially different directions and the light emitting apparatus is capable of adapting and/or conforming to a non-planar surface.

The method may be performed by an apparatus as defined in the first and second aspects.

According to a fifth aspect of the present invention there is provided use of an apparatus according to the first and second aspects in a medical treatment.

Typically, the medical treatment may be a photodynamic therapy or phototherapy.

The present invention may therefore relate to the treatment of cancer (e.g. skin cancer), acne, wrinkles, wound-healing, anti-aging and post-skin laser treatments (e.g. cosmetic applications).

According to a sixth aspect of the present invention there is provided a method of medical treatment using of an apparatus according to the first and second aspects.

The medical treatment may be that of photodynamic therapy or phototherapy.

Moreover, the treatment may be that of the treatment of cancer (e.g. skin cancer), acne, wrinkles, wound-healing, anti-aging and post-skin laser treatments (e.g. cosmetic applications).

### BRIEF DESCRIPTION OF THE DRAWINGS

Embodiments of the present invention will now be described, by way of example only, with reference to the accompanying drawings in which:

FIG. 1 is a representation of a light emitting apparatus according to an embodiment of the present invention;

FIG. 2 is a is a representation of the light emitting apparatus shown in

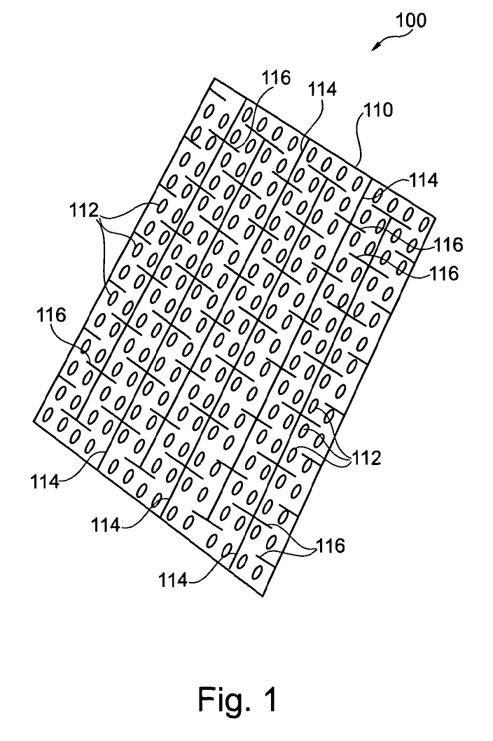

FIG. 1 being flexed;

FIG. 3 is a representation of a light emitting apparatus according to a further embodiment of the present invention which also comprises a flexible substrate upon which conductive traces may be placed;

FIG. 4 is a top view of an organic light emitting diode (OLED) arrangement according to a further embodiment of the present invention;

FIG. 5 is a side view of the organic light emitting diode (OLED) arrangement shown in FIG. 4;

FIG. 6 is a representation on non-linear emission of light from the organic light emitting diode (OLED) arrangement shown in FIGS. 4 and 5;

FIG. 7 is a representation of light emitting apparatus according to a further embodiment of the present invention;

FIG. 8 is a view of the light emitting apparatus shown in FIG. 7 showing patterns of electrode geometry;

FIG. 9 is a representation of a substrate patterning of a conductive trace on a light emitting apparatus according to a further embodiment of the present invention; and

FIG. 10 a representation of a substrate patterning of a conductive trace on a light emitting apparatus according to a yet further embodiment of the present invention.

### BRIEF DESCRIPTION

The present invention therefore relates to an apparatus capable of performing a therapeutic and/or cosmetic treatment on a human or animal patient. The apparatus may be used for any type of medical treatment and, for example, may be used in phototherapy or photodynamic therapy.

FIG. 1 is a representation of a light emitting apparatus according to the present invention, generally designated 100. The light emitting apparatus 100 comprises a flexible substrate 110 made from a flexible PCB or a flexible dielectric upon which conductive traces may be placed. The flexible substrate 110 has a size of about 30 cm×30 cm and a thickness of about 1 mm. As shown in FIG. 1, there is a plurality of light sources 112 embedded/deposited onto the flexible substrate 110. The light sources 112 are attached by solder. FIG. 1 also shows that there are a series of extended substantially parallel cuts 114 extending from one side to almost an extremity of another side. Intersecting the extended substantially parallel cuts 114 are a series of shorter cuts 116. The shorter cuts 116 are in a substantially alternating arrangement on adjacent extended substantially parallel cuts 114. The cuts 114,116 therefore form a grid-like pattern on the flexible substrate 110 but still maintain the flexible substrate 110 in a single-piece of material.

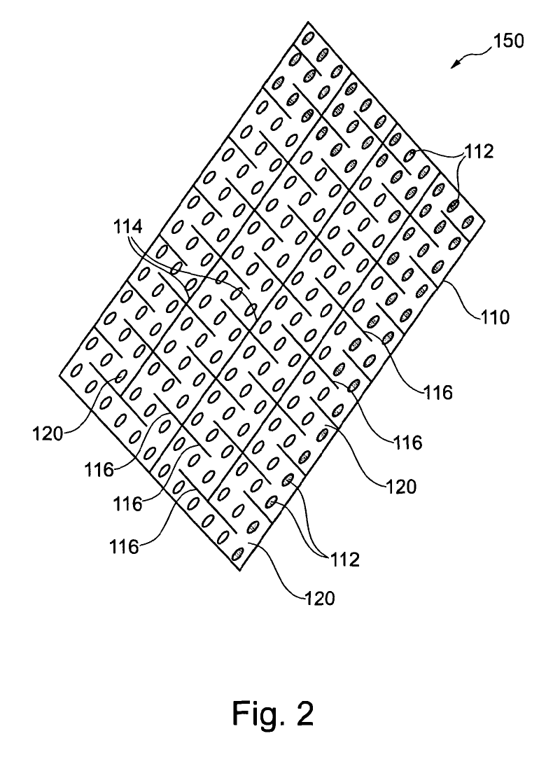

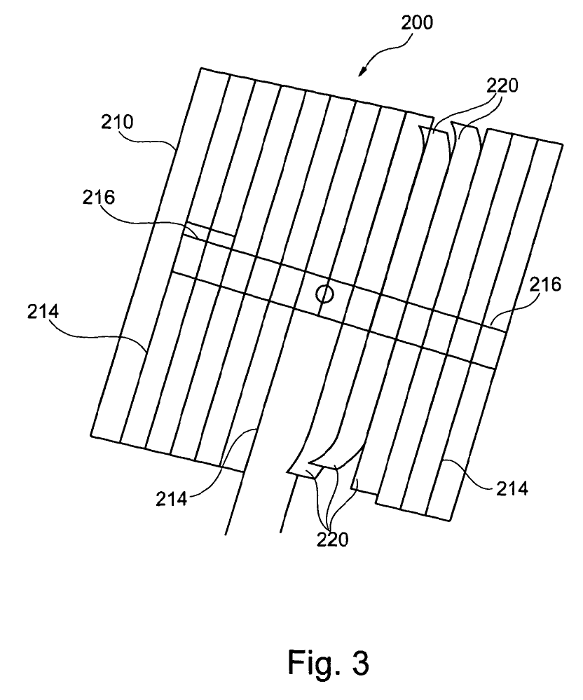

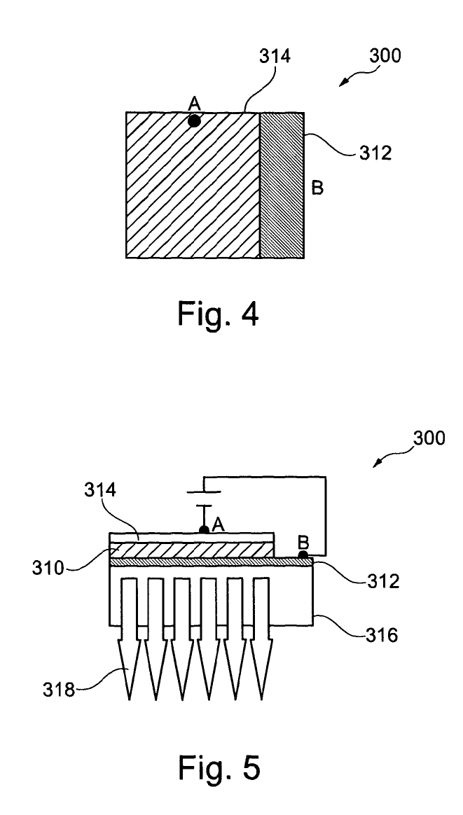

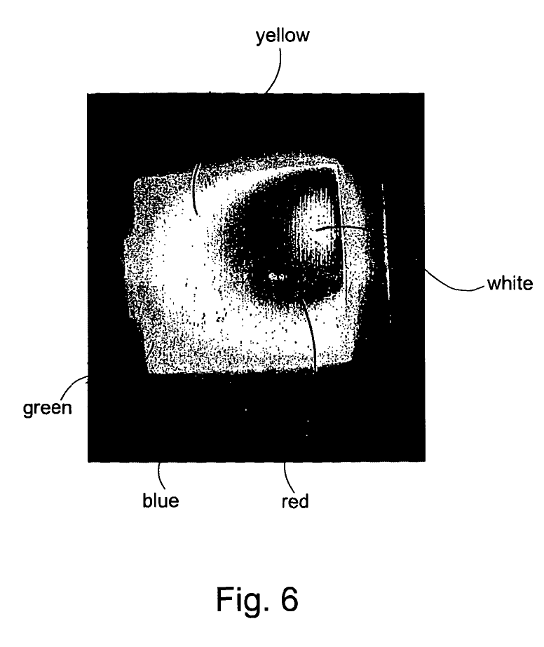

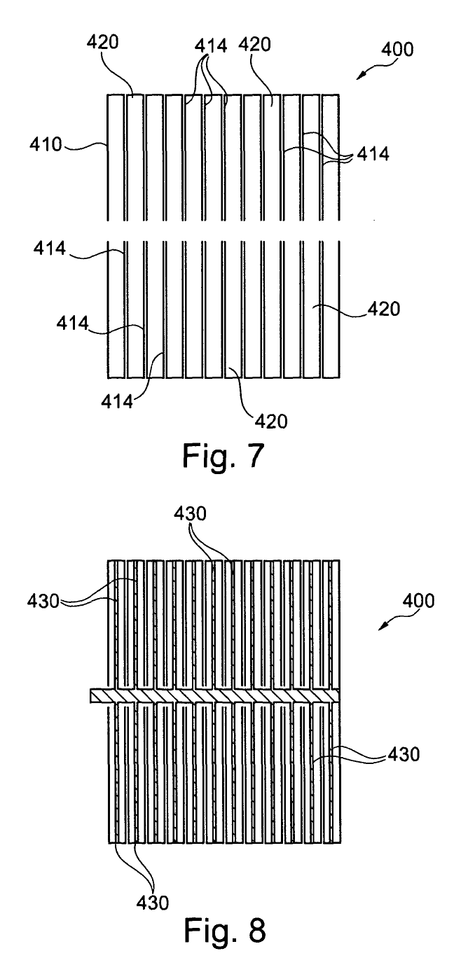

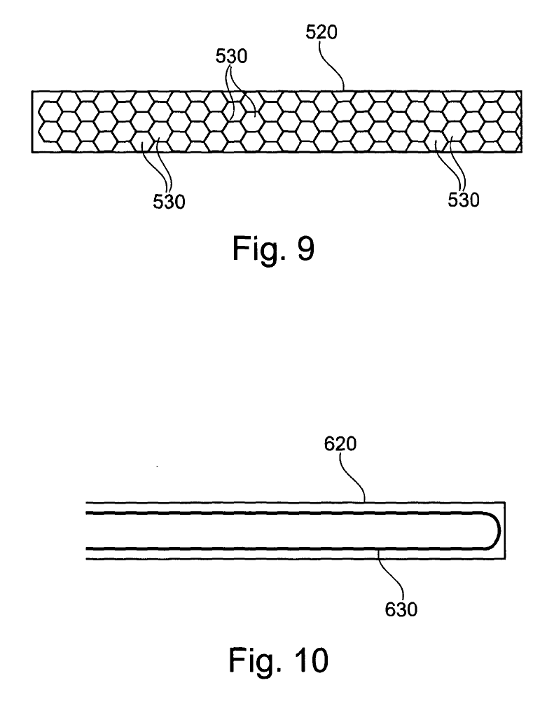

FIG. 2 is a representation of the light emitting apparatus 100 being flexed which shows that the material in between the cuts 114,116 can easily flex and move and thereby adapt and/or conform to a non-planar surface such as on a patient's body. In FIG. 2, the cut-away members 120 in the flexible substrate 110 are shown bending out of the horizontal plane.

FIG. 3 is a representation of a further light emitting apparatus 200 which again comprises a flexible substrate 110 made from a flexible PCB or a flexible dielectric upon which conductive traces may be placed. and a series of extended substantially parallel cuts 214 and a series of shorter cuts 216 which together form substantially ‘L’-shaped cuts in the flexible substrate 210. Light sources for simplicity have been omitted from this diagram. Once again, cut-away members 220 in the flexible substrate 210 are shown bending out of the horizontal plane.

FIGS. 4 and 5 are views of an organic light emitting diode (OLED) generally designated 300. The OLED 300 is a quasi 2D structure that comprises a stack of layers and a substrate 316. In its simplest form, the OLED 300 comprises a thin layer of light emitting polymer 310 sandwiched between two electrodes; an anode and a cathode 312, 314. When a voltage is applied across the stack, light 318 is emitted from the polymer. The cathode 314 in the OLED 300 may be made from low resistivity aluminium. The anode 312 on the other hand is made from a material that has a high sheet resistance. This means that when a voltage is applied across points A and B, the current flows in a non linear manner from A to B and results in non-homogenous light output from the polymer 310. This non-linear emission of light is shown in FIG. 6 which is a false colour image with the red/white area showing greater intensity of emitted light.

The anode 312 and the substrate 316 may be patterned in a similar manner to the patterning shown in FIGS. 1 to 3.

The non-linear emission of light shown in FIG. 6 is well known in the literature and various methods have been proposed to overcome this. The present invention provides a method of introducing thin conductivity traces onto the surface of the anode 312 that distribute current across the surface of the anode 312 thereby overcoming its intrinsically high sheet resistance and inability to distribute current across its surface. This results in more homogenous light output from the polymer 310. The present inventors have found that in a cut/patterned substrate the geometry of the electrode traces must be configured to fit the shape of the ‘legs’.

FIG. 7 is a light emitting apparatus 400 of the present invention formed from a cut/patterned flexible substrate 410. The flexible substrate 410 is made from a flexible PCB or a flexible dielectric upon which conductive traces may be placed. There is shown a series of extended substantially parallel cuts 414 which form cut-away members 420 which are capable of bending and/or flexing out of the horizontal plane.

FIG. 8 is a view of the light emitting apparatus 400 which shows conductive traces 430 extending along the cut-away members 420. The conductive traces 430 extending substantially along the middle section of the cut-away members 420. Although the conductive trace is shown as extending as a straight line other patterns are possible.

FIG. 9 is a view of a further conductive trace pattern 530 on a cut-away member 530 of another light emitting apparatus. The conductive trace pattern 530 is in the form of a honeycomb pattern. However, other conductive trace patterns that form a network or web extending over the cut-away member 530 may be used. It is preferred that the conductive trace pattern in the form of a network or web extends across the whole surface area of the cut-away member.

FIG. 10 is a view of a further conductive trace pattern 630 on a cut-away member 630 of another light emitting apparatus. The conductive trace pattern 630 extends down adjacent to both sides of the cut-away member 630 and then extends adjacent to the end of the cut-away member 630. The conductive trace pattern 630 may therefore be seen as forming a substantially ‘U’-shaped design. The conductive trace pattern 630 may be about 0.5-2 mm away from the edge of the cut-away member 630. By positioning the conductive traces towards and/or adjacent the sides of the cut-away members means that there is more light emitted from the sides than the middle of the cut-away members. This means that when two cut-away members are bent apart from each other, the adjacent surface is still illuminated evenly. Although this method introduces some intra leg variation, it is still minimising light variation across the surface as a whole.

Whilst specific embodiments of the present invention have been described above, it will be appreciated that departures from the described embodiments may still fall within the scope of the present invention. For example, any suitable type of flexible substrate may be used. Furthermore, any suitable type of light source may be used. Moreover, there may be variability in the shape of the weakened areas for particular applications e.g. spiral cuts, in the substrate or circular cuts. In addition, the conductive traces may be in the form of a network or web which extends across the whole surface area of the cut-away member. Alternatively, the conductive trace may be located towards and/or adjacent the sides of the cut-away members which means that there is more light emitted from the sides than the middle of the cut-away members.

1. A light emitting apparatus for use in therapeutic and/or cosmetic treatment, the apparatus comprising:
at least one substrate;
at least one light source disposed on the substrate;
at least a first and second weakened area on the at least one substrate; and
wherein the first and second weakened area have substantially different directions and the light emitting apparatus is capable of adapting and/or conforming to a non-planar surface.

2. A light emitting apparatus for use in therapeutic and/or cosmetic treatment according to claim 1, wherein there are two or more or a plurality of weakened areas.

3-21. (canceled)

22. A light emitting apparatus for use in therapeutic and/or cosmetic treatment, the apparatus comprising:
at least one substrate wherein the at least one substrate is a transparent electrode which is capable of functioning as a light source;
at least a first and second weakened area on the at least one substrate; and
wherein the first and second weakened area have substantially different directions and the light emitting apparatus is capable of adapting and/or conforming to a non-planar surface.

23-37. (canceled)

38. A method of performing a therapeutic and/or cosmetic treatment, the method comprising:
providing at least one substrate;
providing at least one light source disposed on the substrate;
providing at least a first and second weakened area on the at least one substrate; and
wherein the first and second weakened area have substantially different directions and the light emitting apparatus is capable of adapting and/or conforming to a non-planar surface.

39-42. (canceled)

43. A light emitting apparatus for use in therapeutic and/or cosmetic treatment according to claim 1, wherein the weakened areas are thinner areas on the substrate, perforated areas, partially folded areas or cut areas, and wherein the cuts extend from one side of the substrate to the other and therefore form a plurality of slits in the substrate.

44. A light emitting apparatus for use in therapeutic and/or cosmetic treatment according to claim 1, wherein the first and second weakened areas are diverging with respect to each other and, for example, have an angle between them of about 10-170°, about 20-160°, about 30-150°, about 40-140°, about 50-130°, about 60-120°, about 70-110°, about 80-100° or about 90° and are in any suitable form such as in circular and/or spiral cuts.

45. A light emitting apparatus for use in therapeutic and/or cosmetic treatment according to claim 1, wherein there is a series of weakened areas and the weakened areas comprise a series of substantially vertical and substantially horizontal weakened areas, and wherein the series of weakened areas comprises weakened areas spaced apart on an x-axis and substantially horizontal weakened areas spaced apart on a y-axis, and wherein there is a series or plurality of substantially vertical weakened areas which extend substantially the whole length of the substrate but have a remaining part that is not weakened which may be in the form of a single substrate.

46. A light emitting apparatus for use in therapeutic and/or cosmetic treatment according to claim 1, wherein there is a series or plurality of discontinuous substantially horizontal weakened areas which intersect and extend though substantially vertical weakened areas, there is a plurality of substantially vertical weakened areas extending from one side of the substrate to an opposite side and wherein the substantially vertical weakened areas are substantially parallel with one another, and wherein there is a plurality of substantially horizontal weakened areas extending from one side of the substrate to an opposite side.

47. A light emitting apparatus for use in therapeutic and/or cosmetic treatment according to claim 1, wherein there is a plurality of substantially horizontal weakened areas which form a discontinuous ‘dash’-like pattern, there is a series of rows of substantially horizontal weakened areas each in a form of a ‘dash’-like pattern, the material forming the substrate is pliable and is capable of bending, flexing and/or conforming which may be in multiple directions at multiple points on the substrate, and wherein the material forming the substrate is a flexible material (e.g. a flexible polymeric material) such as a thermoplastic elastomer polymer.

48. A light emitting apparatus for use in therapeutic and/or cosmetic treatment according to claim 1, wherein the thickness of the substrate is sufficient to provide support to the at least one light source and is also thick enough to incorporate a diffusing member, and wherein the substrate comprises one, at least one or more or a plurality of light sources located within areas defined by the weakened areas, and wherein areas defined by the weakened areas are substantially rectangular or square shaped with an area of about 0.1 to 4 cm² or about 0.5 to 1 cm².

49. A light emitting apparatus for use in therapeutic and/or cosmetic treatment according to claim 1, wherein the substrate also comprises a diffusing member, and wherein the apparatus also comprises a drug and/or chemical source capable of delivering drugs and/or chemicals to an area of the patient to be treated.

50. A light emitting apparatus for use in therapeutic and/or cosmetic treatment according to claim 22, wherein the weakened area is a series of cuts in the at least one substrate, wherein there is an anode in planar form attached to the substrate and the weakened areas in the form of cuts are formed in both the substrate and the anode, wherein the weakened areas form at least one or more or a plurality of cut-away members which are flexible, and wherein on the cut-away members there is a conductive trace or a plurality of conductive traces which are in the form of a network or web which extends across the whole or substantially the whole surface area of the cut-away member.

51. A light emitting apparatus for use in therapeutic and/or cosmetic treatment according to claim 22, wherein the conductive traces are located towards and/or adjacent the sides of the cut-away members which means that there is more light emitted from the sides than the middle of the cut-away members, and wherein the conductivity traces are thin with a thickness of about 0.1 μm to 5 mm, wherein the conductive trace distributes current across the surface of an anode of the light emitting apparatus (e.g. OLED) thereby overcoming its intrinsically high sheet resistance and inability to distribute current across its surface whereby this results in more homogenous light output from a polymer in the light emitting apparatus, wherein the geometry of the conductive traces are configured to fit the shape of the cut-away members (e.g. the ‘legs’), and wherein the conductive traces extend along the length or substantially a majority of the length of the cut-away members and along the middle or substantially the middle section of the cut-away members.

52. A light emitting apparatus for use in therapeutic and/or cosmetic treatment according claim 22, wherein the conductive trace extends in a straight or substantially straight line, wherein the conductive trace is in the form of a honeycomb pattern, and wherein the conductive trace is in the form a network or web extending over the cut-away member.

53. A light emitting apparatus for use in therapeutic and/or cosmetic treatment according to claim 22, wherein the conductive trace extends down adjacent to both sides of the cut-away member and optionally then extends adjacent to the end of the cut-away member, wherein the conductive trace is in the form of a substantially ‘U’-shaped design, and wherein the conductive trace pattern is adjacent to and about 0.5-2 mm away from the edge of the cut-away member whereby there is more light emitted from the sides than the middle of the cut-away members which means that when two cut-away members are bent apart from each other, an adjacent surface is still illuminated evenly.
